(19) United States
(12) Patent Application Publication
(10) Pub. No.: US 2008/0125703 A1
(43) Pub. Date: May 29, 2008

(54) Breast pump with foot pedal control unit

(76) Inventor: Cathleen Ihm, Farmingdale, NY (US)

Correspondence Address: iPA;STE. 101, 400 GARDEN CITY PLAZA, GARDEN CITY, NY, 11530, US

(21) Appl. No.: 11/604,373

(22) Filed: Nov. 27, 2006

(51) Int. Cl. A61M 1/06

(52) U.S. Cl. 604/74

(57) ABSTRACT

A breast pump comprises an electrically-controlled pump unit and a foot pedal control unit. The electrically-controlled pump unit is configured for being selectively activated, selectively deactivated, selectively operated at a plurality of different suction frequency settings and selectively operated at a plurality of different suction strength settings. The different settings affect a suction pressure generated by the pump unit. The foot pedal control unit includes a suction frequency switch, a suction strength switch and an activation-deactivation switch. The switches are electrically connected to the electrically-controlled pump unit such that a desired one of the suction frequency settings is selectable through manual manipulation of the suction frequency switch, such that a desired one of the suction strength settings is selectable through manual manipulation of the suction strength switch and such that operation of the electrically-controlled pump unit is selectively activated and deactivated through manual manipulation of the activation switch.

### FIELD OF THE DISCLOSURE

The disclosures made herein relate generally to electrical breast pumps and, more particularly, to means for controlling operation of an electrical breast pump.

### BACKGROUND

Feeding a mother's breast milk to her baby is known to offer a number of benefits to the baby. Examples of such benefits include, but are not limited to, enhanced immunization, a preferred balance of nutrients and a nurturing bond between mother and baby. With such benefits, it is not surprising that millions of mothers around the world choose to feed their babies their breast milk.

Because breast-feeding a baby is not always convenient or possible, there are times when it is necessary for a mother to pump her milk for later consumption by a baby. In such cases, a breast pump is utilized to extract the breast milk from the mother's breast, thereby enabling it to be stored for later consumption by her baby. Various types of electric and manual breast pumps are known.

The electric breast pump is known to have widespread appeal by mothers due to its efficiency. Electric breast pumps enable a relatively large volume of breast milk to be pumped in a relatively short period of time. By placing a suction cup assembly of an electric breast pump over a nipple of her breast and activating the pump unit of the electric breast pump, the mother is able readily pump milk from her breast into a reservoir container of the suction cup assembly.

To further enhance efficiency with respect to time, some breast pumps have two suction cup assemblies (i.e., a double breast pump) thereby allowing a mother to simultaneously pump both of her breasts. A drawback of the double breast pump is that it difficult for a mother to simultaneously manipulate the two suction cup assemblies and make necessary adjustments to the hand-operated control unit of conventional double breast pumps. In many cases, a mother must endure pain at the onset of pumping both breasts with a conventional double breast pump because she must set a suction level of the breast pump and/or frequency level of the breast pump to a higher level than initially desired because her hands are thereafter fully occupied with positioning the two suction cup assemblies relative to her nipples and supporting the two suction cup assemblies.

Therefore, a breast pump that eliminates the need to manipulate the various control devices of a breast pump using one's hands would be advantageous, desirable and useful.

### SUMMARY OF THE DISCLOSURE

In one embodiment of the present invention, a breast pump comprises an electrically-controlled pump unit and a foot pedal control unit. The electrically-controlled pump unit is configured for generating a suction pressure. The foot pedal control unit includes a first control device. The first control device is electrically connected to the electrically-controlled pump unit for enabling generation of the suction pressure to be affected through manual manipulation of the first control device.

In another embodiment of the present invention, a breast pump comprises an electrically-controlled pump unit and a foot pedal control unit. The electrically-controlled pump unit is adjustable to a plurality of different pump settings. The different pump settings affect characteristics of a suction pressure generated by the electrically-controlled pump unit. The foot pedal control unit includes at least one control switch. The at least one control switch is electrically connected to the electrically-controlled pump unit for enabling a desired one of the different pump settings to be selected by manual manipulation of the at least one control switch.

In another embodiment of the present invention, a breast pump comprises an electrically-controlled pump unit and a foot pedal control unit. The electrically-controlled pump unit is configured for being selectively activated, selectively deactivated, selectively operated at a plurality of different suction frequency settings and selectively operated at a plurality of different suction strength settings. The different settings affect a suction pressure generated by the pump unit. The foot pedal control unit includes a suction frequency switch, a suction strength switch and an activation-deactivation switch. The switches are electrically connected to the electrically-controlled pump unit such that a desired one of the suction frequency settings is selectable through manual manipulation of the suction frequency switch, such that a desired one of the suction strength settings is selectable through manual manipulation of the suction strength switch and such that operation of the electrically-controlled pump unit is selectively activated and deactivated through manual manipulation of the activation switch.

These and other objects, embodiments, advantages and/or distinctions of the present invention will become readily apparent upon further review of the following specification, associated drawings and appended claims.

### BRIEF DESCRIPTION OF THE DRAWINGS

FIG. 1 shows a breast pump in accordance with the present invention.

FIG. 2 is a block diagram of the breast pump shown in FIG. 1.

FIG. 3 is a top view of a foot pedal control unit of the breast pump shown in FIG. 1.

### DETAILED DESCRIPTION OF THE DRAWING FIGURES

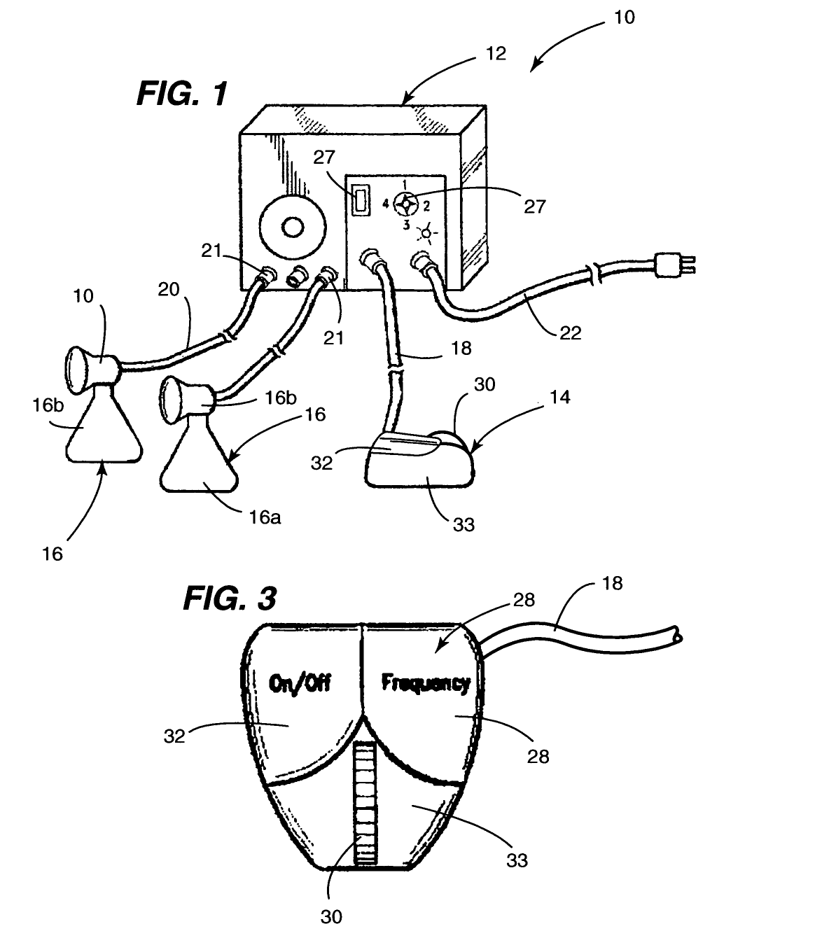

FIG. 1 shows an embodiment of a breast pump in accordance with the present invention, which is referred to herein as the breast pump 10. The breast pump 10 includes an electrically-controlled pump unit 12, a foot pedal control unit 14, and two suction cup assemblies 16. Each one of the suction cup assemblies 16 includes a reservoir 16a and a suction cup 16b. The suction cup 16b typically includes an apparatus (e.g., diaphragm valve device) for jointly controlling application of a suction pressure (e.g., negative pressure commonly referred to as a vacuum) and delivery of breast milk into the reservoir 16a.

The foot pedal control unit 14 is connected to the pump unit 12 by an electrical cable 18 and the two suction cup assemblies 16 are each connected to the pump unit 12 by a respective pneumatic tube 20. Each respective pneumatic tube 20 is connected to a respective suction pressure port 21 of the pump unit 12. In this manner, the two suction cup assemblies 16 are simultaneously attachable to the pump unit 12 for enabling the suction pressure generated by the suction pump apparatus 24 to be simultaneously applied to the two suction cup assemblies 16. The pump unit 12 includes a power cord 22 for supplying electrical power thereto. In other embodiments, the pump unit 12 is powered by a battery power supply.

Referring now to FIG. 2, the pump unit 12 includes a suction pump apparatus 24 and a control module 26 electrically connected to the suction pump apparatus 24. In one embodiment, the suction pump apparatus 24 includes an electric vacuum pump 24 configured for generating a suction pressure (i.e., a negative pressure), which is provided at the suction pressure ports 21, and the control module 26 includes electronic and/or electrical components configured for enabling the suction pump apparatus 24 to be selectively activated, selectively deactivated, selectively operated at a plurality of different suction frequency settings and selectively operated at a plurality of different suction strength settings. A skilled person will appreciate that there are numerous known circuits suitable for providing such selective activation, selective deactivation, different suction frequencies and different suction strengths. Suction frequency relates to the frequency at which suction pressure pulses are generated by the suction pump apparatus 24 and suction strength relates to the magnitude of the pressure pulses. Accordingly, the different settings affect the suction pressure generated by the pump unit. The pump unit 12 includes control elements 27 integral therewith for enabling auxiliary control of at least one of such selective activation, selective deactivation, different suction frequencies and different suction strengths via manual manipulation of such control elements (e.g., when use of the foot pedal control unit is not necessary and/or desirable).

Now referring to FIGS. 1-3, the foot pedal control unit 14 includes a suction frequency switch 28, a suction strength switch 30 and an activation-deactivation switch 32. The suction frequency switch 28, the suction strength switch 30 and the activation-deactivation switch 32 include each include a control portion accessible at an exterior surface of a housing 33 of the foot pedal control unit 14. The switches (28-32) are electrically connected to the pump unit 12 via the electrical cable 18. The control module 26 and the switches (28-32) of the foot pedal control unit 14 are jointly configured for enabling a desired one of the suction frequency settings to be selected through manual manipulation of the suction frequency switch 28, for enabling a desired one of the suction strength settings to be selected through manual manipulation of the suction strength switch 30 and for enabling operation of the pump unit 12 to be selectively activated and deactivated through manual manipulation of the activation switch 32. For example, the control module 26 interprets signals from the switches (28-32) and outputs corresponding voltage/control signals to the suction pump apparatus 24.

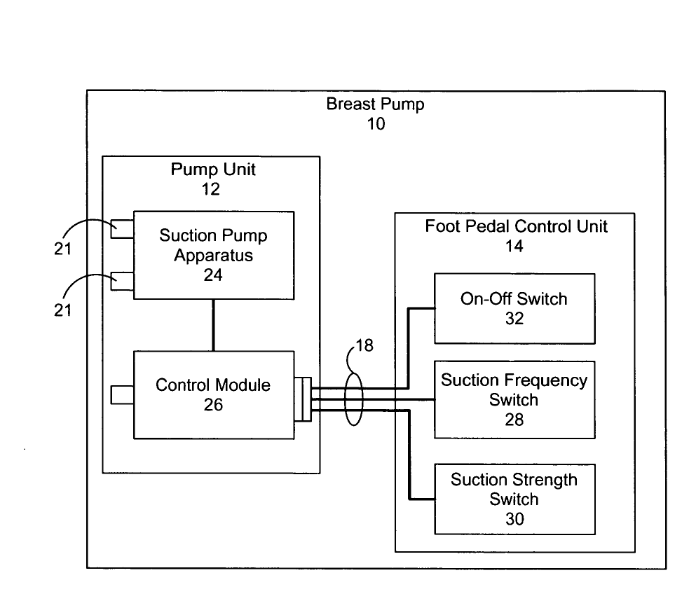

In one embodiment, as shown in FIG. 3, the suction frequency switch 28 is a push button switch, the suction strength switch 30 is a rotary wheel switch and the activation-deactivation switch 32 is a push button switch. The suction frequency switch 28 and the activation-deactivation switch 32 are in side-by-side relationship. The suction strength switch 30 is offset from the suction frequency switch 28 and the activation-deactivation switch 32 with a radial axis of the suction strength switch 30 extending between the suction frequency switch 28 and the activation-deactivation switch 32. A skilled person will appreciate that the suction frequency switch 28, the suction strength switch 30 and the activation-deactivation switch 32 are each a respective embodiment of a control device in accordance with the present invention, which each allow for adjustment of a operating condition (e.g., setting) of the pump unit 12 through manual manipulation thereof. It is disclosed herein that other types of control devices may be implemented in place of those shown.

The breast pump 10 disclosed herein is one embodiment of a breast pump in accordance of the present invention. Breast pumps in accordance with the present invention enable a woman to control activation-deactivation, suction frequency and/or suction strength using one of her feet. Such ability to control activation-deactivation, suction frequency and/or suction strength provides convenience relative to conventional breast pumps, particularly double breast pumps, by allowing the woman to control some or all of the various settings of the breast pump with one of her feet while leaving her hands free to keep the suction cup assemblies properly positioned and supported at all times during breast pumping. This is in contract to conventional double breast pumps that require the user to let go of one or both suction cup assemblies to facilitate manual manipulation of the control devices of the pump unit.

In the preceding detailed description, reference has been made to the accompanying drawings that form a part hereof, and in which are shown by way of illustration specific embodiments in which the present invention may be practiced. These embodiments, and certain variants thereof, have been described in sufficient detail to enable those skilled in the art to practice embodiments of the present invention. It is to be understood that other suitable embodiments may be utilized and that logical, mechanical, chemical and electrical changes may be made without departing from the spirit or scope of such inventive disclosures. To avoid unnecessary detail, the description omits certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A breast pump, comprising:
an electrically-controlled pump unit configured for generating a suction pressure; and
a foot pedal control unit including a first control device, wherein the first control device is electrically connected to the electrically-controlled pump unit for enabling generation of the suction pressure to be affected through manual manipulation of the first control device.

2. The breast pump of claim 1 wherein the electrically-controlled pump unit includes two suction pressure ports whereby two suction cup assemblies are attachable to the electrically-controlled pump unit for enabling the suction pressure generated by the electrically-controlled pump unit to be simultaneously applied to said two suction cup assemblies.

3. The breast pump of claim 1 wherein said manual manipulation of the first control device adjusts at least one of activation of the electrically-controlled pump unit, a suction frequency setting of the electrically-controlled pump unit and a suction strength setting of the electrically-controlled pump unit.

4. The breast pump of claim 3 wherein the electrically-controlled pump unit includes two suction pressure ports whereby two suction cup assemblies are attachable to the electrically-controlled pump unit for enabling the suction pressure generated by the electrically-controlled pump unit to be simultaneously applied to said two suction cup assemblies.

5. The breast pump of claim 1 wherein:
the electrically-controlled pump unit is configured for being selectively operated at a plurality of different suction frequency settings and selectively operated at a plurality of different suction strength settings;
a desired one of said suction frequency settings is selectable through manual manipulation of the first control device.
the foot pedal control unit includes a second control device electrically connected to the electrically-controlled pump unit; and
a desired one of said suction strength settings is selectable through manual manipulation of the second control device.

6. The beast pump of claim 5 wherein:
the electrically-controlled pump unit is configured for being selectively activated and deactivated;
the foot pedal control unit includes a third control device electrically connected to the electrically-controlled pump unit; and
operation of the electrically-controlled pump unit is selectively activated and deactivated through manual manipulation of the third control device.

7. The breast pump of claim 6 wherein:
the first control device is a first push button switch;
the second control device is a rotary wheel switch;
the third control device is a second push button switch;
the first push button switch and the second push button switch are in side-by-side relationship; and
a radial axis of the rotary wheel switch extends between the first and second push button switches.

8. The breast pump of claim 6 wherein:
the electrically-controlled pump unit includes two suction pressure ports whereby two suction cup assemblies are attachable to the electrically-controlled pump unit for enabling the suction pressure generated by the electrically-controlled pump unit to be simultaneously applied to said two suction cup assemblies.

9. A breast pump, comprising:
an electrically-controlled pump unit adjustable to a plurality of different pump settings, wherein said different pump settings affect characteristics of a suction pressure generated by the electrically-controlled pump unit; and
a foot pedal control unit including at least one control switch, wherein said at least one control switch is electrically connected to the electrically-controlled pump unit for enabling a desired one of said different pump settings to be selected by manual manipulation of said at least one control switch.

10. The breast pump of claim 9 wherein the electrically-controlled pump unit includes two suction pressure ports whereby two suction cup assemblies are attachable to the electrically-controlled pump unit for enabling the suction pressure generated by the electrically-controlled pump unit to be simultaneously applied to said two suction cup assemblies.

11. The breast pump of claim 9 wherein:
said different pump settings include a plurality of different suction frequency settings and a plurality of different suction strength settings.

12. The breast pump of claim 11 wherein the electrically-controlled pump unit includes two suction pressure ports whereby two suction cup assemblies are attachable to the electrically-controlled pump unit for enabling the suction pressure generated by the electrically-controlled pump unit to be simultaneously applied to said two suction cup assemblies.

13. The breast pump of claim 9 wherein:
the electrically-controlled pump unit is configured for being selectively operated at a plurality of different suction frequency settings and selectively operated at a plurality of different suction strength settings;
the foot pedal control unit includes two control switches electrically connected to the electrically-controlled pump unit;
a desired one of said suction frequency settings is selectable through manual manipulation of a first one of said control switches; and
a desired one of said suction strength settings is selectable through manual manipulation of a second one of said control switches.

14. The beast pump of claim 13 wherein:
the electrically-controlled pump unit is configured for being selectively activated and deactivated;
the foot pedal control unit includes three control switches electrically connected to the electrically-controlled pump unit; and
operation of the electrically-controlled pump unit is selectively activated and deactivated through manual manipulation of a third one of said control switches.

15. The breast pump of claim 14 wherein:
the first one of said control switches is a push button switch;
the second one of said control switches is a rotary wheel switch;
the third one of said control switches is a push button switch;
the first one of said control switches and the third one of said control switches are in side-by-side relationship; and
a radial axis of the second one of said control switches extends between the first one of said control switches and the third one of said control switches.

16. The breast pump of claim 14 wherein:
the electrically-controlled pump unit includes two suction pressure ports whereby two suction cup assemblies are attachable to the electrically-controlled pump unit for enabling the suction pressure generated by the electrically-controlled pump unit to be simultaneously applied to said two suction cup assemblies.

17. A breast pump, comprising:
an electrically-controlled pump unit configured for being selectively activated, selectively deactivated, selectively operated at a plurality of different suction frequency settings and selectively operated at a plurality of different suction strength settings, wherein said different settings affect a suction pressure generated by the pump unit; and
a foot pedal control unit including a suction frequency switch, a suction strength switch and an activation-deactivation switch, wherein said switches are electrically connected to the electrically-controlled pump unit such that a desired one of said suction frequency settings is selectable through manual manipulation of the suction frequency switch, such that a desired one of said suction strength settings is selectable through manual manipulation of the suction strength switch and such that operation of the electrically-controlled pump unit is selectively activated and deactivated through manual manipulation of the activation switch.

18. The breast pump of claim 17 wherein the electrically-controlled pump unit includes two suction pressure ports whereby two suction cup assemblies are attachable to the electrically-controlled pump unit for enabling the suction pressure generated by the electrically-controlled pump unit to be simultaneously applied to said two suction cup assemblies.

19. The breast pump of claim 17 wherein:
the first control switch is a push button switch;
the second control switch is a rotary wheel switch;
the third control switch is a push button switch;
the first and third control switches are in side-by-side relationship; and
a radial axis of the second control switch extends between the first and third control switches.

20. The breast pump of claim 19 wherein:
the electrically-controlled pump unit includes two suction pressure ports whereby two suction cup assemblies are attachable to the electrically-controlled pump unit for enabling the suction pressure generated by the electrically-controlled pump unit to be simultaneously applied to said two suction cup assemblies.
